(19) United States
(12) Patent Application Publication
(10) Pub. No.: US 2002/0081608 A1
(43) Pub. Date: Jun. 27, 2002

(54) Tumor associated proteins

(76) Inventors: Liu-Ying Luo, Toronto (CA); Eleftherios P. Diamandis, Toronto (CA)

Correspondence Address: MERCHANT & GOULD PC, P.O. BOX 2903, MINNEAPOLIS, MN, 55402-0903, US

(21) Appl. No.: 09/909,147

(22) Filed: Jul. 19, 2001

Related U.S. Application Data
(60) Provisional application No. 60/219,674, filed on Jul. 21, 2000.
(60) Provisional application No. 60/273,451, filed on Mar. 5, 2001.

(51) Int. Cl. C12Q 1/68; G01N 33/574; C07H 21/04; C12P 21/02; C12N 5/06; C07K 14/435

(52) U.S. Cl. 435/6; 435/7.23; 435/325; 435/320.1; 435/69.3; 530/350; 536/23.5

(57) ABSTRACT

The invention relates to the identification of tumor associated proteins, novel nucleic acid molecules encoding tumor associated proteins and proteins encoded by such nucleic acid molecules; and uses of the tumor associated proteins.

### FIELD OF THE INVENTION

The invention relates to the identification of tumor associated proteins, novel nucleic acid molecules encoding tumor associated proteins and proteins encoded by such nucleic acid molecules; and uses of the tumor associated proteins.

### BACKGROUND OF THE INVENTION

Cancer is one of the leading causes of death in humans. In North America, it is estimated that one in four people will die of cancer. The etiology of cancer is still not clear. Although some cancers have specific etiology, in general, it is thought that both genetic factors and environmental carcinogens contribute to disease pathogenesis.

In cancer, it is now known that certain cellular components may act as immunogens, thus triggering autoimmune responses. The underlying mechanisms include (a) failure of self-tolerance of the immune system; (b) aberrant expression level of certain cellular components; and, (c) expression of mutated forms of certain cellular components. The circulating autoantibodies to such proteins have potential for cancer diagnosis, monitoring or prognosis, and for developing therapeutic cancer vaccines (2-5).

Autoimmune reactions have been described frequently in ovarian cancer. Two proteins have been investigated intensively, CA-125 and p53 (6-13). High titers of CA-125 and p53 autoantibodies have been detected in a significant proportion of ovarian cancer patients.

### SUMMARY OF THE INVENTION

Applicants have identified cellular components that may trigger immune responses in cancer patients. In particular, Applicants screened an ovarian carcinoma cDNA expression library with ascites from ovarian cancer patients and identified cellular proteins that trigger autoantibody production. (See Table 1.) The proteins include ribosomal protein S18 (Genbank X69150), heat shock protein (90 kda) (Genbank M16660), JK-recombination signal binding protein (Genbank (L07872), ribonucleoprotein H1 (Genbank L22009), RAN binding protein 7 (AF098799), TG-interacting factor (Genbank X89750), eukaryotic translation initiation factor 3, p40 (Genbank U54559), human amyloid percursor protein-binding protein 1 (Genbank U50939), ribosomal protein L8 (NM000973), CDC23 (Cell division 23) (Genbank NM004661), IQ motif containing GTPase activating protein 1 (IQGAP1) (Genbank NM003870); and ribosomal protein L3 (Genbank NM000967).

A novel nucleic acid molecule was also identified by Applicants in the immunoscreen of the ovarian carcinoma cDNA library. The novel nucleic acid molecule was found to be down-regulated in testicular tumors, in comparison to adjacent normal tissues. The present inventors also identified two alternatively spliced forms encoding for slightly different proteins. The novel proteins encoded by the nucleic acid molecules described herein are referred to as “DTC” or “DTC Protein”. The longer spliced form and the shorter spliced form are more particularly referred to herein as the “A” form, and the “B” form, respectively. The nucleic acid molecules encoding the proteins are referred to as “dtc”.

The cellular proteins that trigger autoantibody production identified in the immunoscreen including the proteins referenced above and DTC Related Proteins described herein are referred to herein as “tumor associated protein(s)”.

Broadly stated the present invention relates to an isolated nucleic acid molecule of at least 30 nucleotides which hybridizes to one or more of SEQ. ID. NO. 1, 3, and 5 to 21, or the complement of one or more of SEQ ID NO. 1, 3, and 5 to 21, under stringent hybridization conditions.

The invention also contemplates a nucleic acid molecule comprising a sequence encoding a truncation of a DTC Protein, an analog, or a homolog of a DTC Protein or a truncation thereof. (DTC Protein and truncations, analogs and homologs of DTC Protein are also collectively referred to herein as “DTC Related Proteins”).

The nucleic acid molecules of the invention may be inserted into an appropriate expression vector, i.e. a vector that contains the necessary elements for the transcription and translation of the inserted coding sequence. Accordingly, recombinant expression vectors adapted for transformation of a host cell may be constructed which comprise a nucleic acid molecule of the invention and one or more transcription and translation elements linked to the nucleic acid molecule.

The recombinant expression vector can be used to prepare transformed host cells expressing DTC Related Proteins. Therefore, the invention further provides host cells containing a recombinant molecule of the invention. The invention also contemplates transgenic non-human mammals whose germ cells and somatic cells contain a recombinant molecule comprising a nucleic acid molecule of the invention, in particular one which encodes an analog of a DTC Protein, or a truncation of a DTC Protein.

The invention further provides a method for preparing DTC Related Proteins utilizing the purified and isolated nucleic acid molecules of the invention. In an embodiment a method for preparing a DTC Related Protein is provided comprising (a) transferring a recombinant expression vector of the invention into a host cell; (b) selecting transformed host cells from untransformed host cells; (c) culturing a selected transformed host cell under conditions which allow expression of the DTC Related Protein; and (d) isolating the DTC Related Protein.

The invention further broadly contemplates an isolated DTC Protein comprising an amino acid sequence of SEQ.ID.NO. 2 or 4.

The DTC Related Proteins of the invention may be conjugated with other molecules, such as proteins, to prepare fusion proteins. This may be accomplished, for example, by the synthesis of N-terminal or C-terminal fusion proteins.

The invention further contemplates antibodies having specificity against an epitope of a tumor associated protein described herein, in particular a DTC Related Protein of the invention. Antibodies may be labeled with a detectable substance and used to detect proteins of the invention in tissues and cells. Antibodies may have particular use in therapeutic applications, for example to react with tumor cells, and in conjugates and immunotoxins as target selective carriers of various agents which have antitumor effects including chemotherapeutic drugs, toxins, immunological response modifiers, enzymes, and radioisotopes.

The invention also permits the construction of nucleotide probes that are unique to the nucleic acid molecules encoding tumor associated proteins, in particular a DTC Related Protein. In an embodiment, the invention relates to a probe comprising a nucleic acid molecule of the invention, or a nucleic acid sequence encoding a tumor associated protein described herein, or a part thereof. The probe may be labeled, for example, with a detectable substance and it may be used to select from a mixture of nucleotide sequences a nucleic acid molecule encoding a tumor associated protein, in particular a DTC Related Protein. A probe may be used to mark tumors, in particular ovarian and testicular tumors.

The invention also provides antisense nucleic acid molecules e.g. by production of a mRNA or DNA strand in the reverse orientation to a sense molecule. An antisense nucleic acid molecule may be used to suppress the growth of a tumor associated protein (e,g. DTC Protein) expressing (e.g. cancerous) cell.

The invention still further provides a method for identifying a substance which binds to a tumor associated protein (e.g. DTC Related Protein) comprising reacting the protein with at least one substance which potentially can bind with the protein, under conditions which permit the formation of complexes between the substance and protein and detecting binding. Binding may be detected by assaying for complexes, for free substance, or for non-complexed protein. The invention also contemplates methods for identifying substances that bind to other intracellular proteins that interact with a tumor associated protein (e.g. a DTC Related Protein). Methods can also be utilized which identify compounds which bind to tumor associated protein gene regulatory sequences (e.g. promoter sequences of a DTC gene).

Still further the invention provides a method for evaluating a compound for its ability to modulate the biological activity of a tumor associated protein (e.g. DTC Related Protein). For example a substance which inhibits or enhances the interaction of the protein and a substance which binds to the protein may be evaluated. In an embodiment, the method comprises providing a known concentration of a tumor associated protein (e.g. DTC Related Protein), with a substance which binds to the protein and a test compound under conditions which permit the formation of complexes between the substance and protein, and removing and/or detecting complexes.

Compounds which modulate the biological activity of a tumor associated protein of the invention may also be identified using the methods of the invention by comparing the pattern and level of expression of the protein in tissues and cells, in the presence, and in the absence of the compounds.

Antibodies and antisense nucleic acid molecules, substances and compounds identified using the methods of the invention, and tumor associated proteins of the invention may be used to modulate the biological activity of a tumor associated protein, in particular a DTC Related Protein, and they may be used in the treatment of conditions such as cancer (particularly testicular and ovarian cancer) in a patient. Accordingly, the substances and compounds may be formulated into compositions for administration to individuals suffering from disorders such as cancer (particularly testicular and ovarian cancer) in a patient. In particular, the antibodies, antisense nucleic acid molecules, substances and compounds may be used to treat patients who have a tumor associated protein in, or on, their cancer cells.

Therefore, the present invention also relates to a composition comprising one or more of a tumor associated protein described herein, a peptide thereof, or a substance or compound identified using the methods of the invention, and a pharmaceutically acceptable carrier, excipient or diluent. A method for treating or preventing a disorder such as cancer (particularly testicular and ovarian cancer) in a patient is also provided comprising administering to a patient in need thereof, a tumor associated protein ((e.g a DTC Related Protein), or a composition of the invention.

Another aspect of the invention is the use of a tumor associated protein (e.g. DTC Protein), peptides derived therefrom, or chemically produced (synthetic) peptides, or any combination of these molecules, for use in the preparation of vaccines to prevent cancer and/or to treat cancer, in particular to prevent and/or treat cancer in patients who have a tumor associated protein detected on their cells. These vaccine preparations may also be used to prevent patients from having tumors, in particular ovarian or testicular tumors, prior to their occurrence.

The invention broadly contemplates vaccines for stimulating or enhancing in a subject to whom the vaccine is administered production of antibodies directed against a tumor associated protein (e.g. DTC Protein).

The invention also provides a method for stimulating or enhancing in a subject production of antibodies directed against a tumor associated protein (e.g. DTC Protein). The method comprises administering to the subject a vaccine of the invention in a dose effective for stimulating or enhancing production of the antibodies.

The invention further provides methods for treating, preventing, or delaying recurrence of cancer. The methods comprise administering to the subject a vaccine of the invention in a dose effective for treating, preventing, or delaying recurrence of cancer.

In other embodiments, the invention provides a method for identifying inhibitors of a DTC Related Protein interaction, comprising

(a) providing a reaction mixture including the DTC Related Protein and a substance that binds to the DTC Related Protein, or at least a portion of each which interact;

(b) contacting the reaction mixture with one or more test compounds;

(c) identifying compounds which inhibit the interaction of the DTC Related Protein and substance.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a DTC Related Protein and a substance that binds to the protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows an example of a positive clone (indicated by the arrow) identified by immunoscreening of an ovarian carcinoma cDNA expression library, probed with ovarian cancer ascites.

FIG. 2 shows the full-length cDNA sequence of the novel gene, alternatively spliced forms, and their open reading frames. The bold sequences in the 3′ end indicate the polyadenylation signal (AATAAA) and the polyA tail. The underlined DNA and protein sequences denote the sequences which are present in the alternatively spliced form A, but not in form B. The DNA and the protein sequences unique in form B are shown in italic and bold letters. Asterisks indicate stop codons.

FIG. 3 is a Northern blot analysis of the novel gene to determine the transcript size in various human tissues. The position of the RNA marker is indicated on the left side of the picture.

FIG. 4 shows tissue expression of the novel gene, determined by RT-PCR. The various human tissues are shown on the top of each lane and the DNA marker is indicated on the left side of the picture. Note the alternatively spliced form in brain, cerebellum, and spinal cord.

FIG. 5 shows the genomic organization and partial nucleotide sequence of the novel gene. The promoter and intron sequences are shown with lower case letters. Exon sequences are in capital and bold letters. Dotted lines represent sequence that is not shown. The full sequences of some introns are not shown. The intron/exon number and length are as indicated. The SP1, CREB binding sites and splice junction donor/acceptor sites are underlined.

FIG. 6. Chromosomal localization of the novel gene by fluorescence in situ hybridization. This gene is localized to chromosome 4p11.

FIG. 7 shows a comparison of the expression level of the novel gene in testicular tumors and their adjacent normal tissues with RT-PCR. The horizontal lines on the top of the picture indicate the tissue pairs. The first lane represents the normal tissue and the second lane is the paired tumor specimen. The arrows and “N” above the horizontal lines indicate whether the expression is down-regulated (⇓) or no change (N), respectively.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

1. Nucleic Acid Molecules of the Invention

As hereinbefore mentioned, the invention provides an isolated nucleic acid molecule having a sequence encoding a DTC Protein. The term “isolated” refers to a nucleic acid substantially free of cellular material or culture medium when produced by recombinant DNA techniques, or chemical reactants, or other chemicals when chemically synthesized. An “isolated” nucleic acid may also be free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double stranded or single stranded. In an embodiment, a nucleic acid molecule encodes a DTC Protein comprising an amino acid sequence of SEQ.ID.NO. 2 or 4, preferably a nucleic acid molecule comprising a nucleic acid sequence of SEQ.ID.NO. 1 or 3.

In an embodiment, the invention provides an isolated nucleic acid molecule which comprises:

(i) a nucleic acid sequence encoding a protein having substantial sequence identity with an amino acid sequence of SEQ. ID. NO. 2 or 4;

(ii) a nucleic acid sequence encoding a protein comprising an amino acid sequence of SEQ. ID. NO. 2or4;

(iii) nucleic acid sequences complementary to (i) or (ii);

(iv) a degenerate form of a nucleic acid sequence of (i) or (ii);

(v) a nucleic acid sequence capable of hybridizing under stringent conditions to a nucleic acid sequence in (i), (ii) or (iii);

(vi) a nucleic acid sequence encoding a truncation, an analog, an allelic or species variation of a protein comprising an amino acid sequence of SEQ. ID. NO. 2 or 4; or

(vii) a fragment, or allelic or species variation of (i), (ii) or (iii).

Preferably, a purified and isolated nucleic acid molecule of the invention comprises:

(i) a nucleic acid sequence comprising the sequence of one or more of SEQ.ID.NO. 1, 3, and 5 to 21 wherein T can also be U;

(ii) nucleic acid sequences complementary to (i), preferably complementary to the full nucleic acid sequence of one or more of SEQ.ID.NO. 1, 3, and 5 to 21;

(iii) a nucleic acid capable of hybridizing under stringent conditions to a nucleic acid of (i) or (ii) and preferably having at least 18, 20, 25, 30, 35, or 40 nucleotides; or

(iv) a nucleic acid molecule differing from any of the nucleic acids of (i) to (iii) in codon sequences due to the degeneracy of the genetic code.

The invention includes nucleic acid sequences complementary to a nucleic acid encoding a DTC Protein comprising an amino acid sequence of SEQ.ID.NO. 2 or 4, preferably the nucleic acid sequences complementary to a full nucleic acid sequence of SEQ.ID. NO 1 or 3.

The invention includes nucleic acid molecules having substantial sequence identity or homology to nucleic acid sequences of the invention or encoding proteins having substantial identity or similarity to the amino acid sequence of SEQ.ID.NO. 2 or 4. Preferably, the nucleic acids have substantial sequence identity for example at least 45%, 50%, 55%, 60%, 65%, 70%, 75%, 80%, or 85% nucleic acid identity; more preferably 90% nucleic acid identity; and most preferably at least 95%, 96%, 97%, 98%, or 99% sequence identity. “Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Acadmeic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Isolated nucleic acid molecules encoding a DTC Protein, and having a sequence which differs from a nucleic acid sequence of the invention due to degeneracy in the genetic code are also within the scope of the invention. Such nucleic acids encode functionally equivalent proteins (e.g. a DTC Protein) but differ in sequence from the sequence of a DTC Protein due to degeneracy in the genetic code. As one example, DNA sequence polymorphisms within the nucleotide sequence of a DTC Protein may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a DTC Protein. These amino acid polymorphisms are also within the scope of the present invention.

Another aspect of the invention provides a nucleic acid molecule which hybridizes under stringent conditions, preferably high stringency conditions to a nucleic acid molecule which comprises a sequence which encodes a DTC Protein having an amino acid sequence of SEQ.ID.NO. 2 or 4. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

It will be appreciated that the invention includes nucleic acid molecules encoding a DTC Related Protein including truncations of a DTC Protein, and analogs of a DTC Protein as described herein. The truncated nucleic acids or nucleic acid fragments may correspond to a sequence of one of SEQ ID Nos. 5 to 21 inclusive. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labelled nucleic acid probe based on all or part of a nucleic acid sequence of the invention. The labeled nucleic acid probe is used to screen an appropriate DNA library (e.g. a cDNA or genomic DNA library). For example, a cDNA library can be used to isolate a cDNA encoding a DTC Related Protein by screening the library with the labeled probe using standard techniques. Alternatively, a genomic DNA library can be similarly screened to isolate a genomic clone encompassing a gene encoding a DTC Related Protein. Nucleic acids isolated by screening of a cDNA or genomic DNA library can be sequenced by standard techniques.

An isolated nucleic acid molecule of the invention which is DNA can also be isolated by selectively amplifying a nucleic acid encoding a DTC Related Protein using the polymerase chain reaction (PCR) methods and cDNA or genomic DNA. It is possible to design synthetic oligonucleotide primers from the nucleotide sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a DTC Related Protein into an appropriate vector which allows for transcription of the cDNA to produce an RNA molecule which encodes a DTC Related Protein. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a DTC Related Protein can be accomplished by expressing the cDNA in an appropriate host cell by standard techniques, and testing the expressed protein in the methods described herein. A cDNA encoding a DTC Related Protein can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a DTC Related Protein may be determined using computer software designed for the purpose, such as PC/Gene (IntelliGenetics Inc., Calif.). The intron-exon structure and the transcription regulatory sequences of a gene encoding a DTC Related Protein may be confirmed by using a nucleic acid molecule of the invention encoding a DTC Related Protein to probe a genomic DNA clone library. Regulatory elements can be identified using standard techniques. The function of the elements can be confirmed by using these elements to express a reporter gene such as the lacZ gene that is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art. In an embodiment, regulatory sequences of a nucleic acid molecule of the invention comprise the sequences of SEQ ID NOs. 22 or 23.

In a particular embodiment of the invention, the nucleic acid molecules isolated using the methods described herein are mutant dtc gene alleles. The mutant alleles may be isolated from individuals either known or proposed to have a genotype which contributes to the symptoms of a disorder involving a DTC Related Protein. Mutant alleles and mutant allele products may be used in therapeutic and diagnostic methods described herein. For example, a cDNA of a mutant dtc gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal dtc gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant dtc allele. Gene products made by the putatively mutant tissue may be expressed and screened, for example using antibodies specific for a DTC Related Protein as described herein. Library clones identified using the antibodies can be purified and subjected to sequence analysis.

The sequence of a nucleic acid molecule of the invention, or a fragment of the molecule, may be inverted relative to its normal presentation for transcription to produce an antisense nucleic acid molecule. An antisense nucleic acid molecule may be constructed using chemical synthesis and enzymatic ligation reactions using procedures known in the art.

2. Proteins of the Invention

An amino acid sequence of a DTC Protein comprises a sequence as shown in SEQ.ID.NO. 2 or 4. A sequence of an A form of a DTC Protein is shown in SEQ ID NO. 2, and a sequence of a B form of a DTC Protein is shown in SQEQ ID NO 4. The A form of the protein is expressed in many tissues including testis, brain, placenta, ovary, prostate, and mammary gland. The short B form of the protein is restricted to the central nervous system including brain, cerebellum, and spinal cord.

In addition to proteins comprising an amino acid sequence of SEQ.ID.NO. 2 or 4, the proteins of the present invention include truncations of a DTC Protein, analogs of a DTC Protein, and proteins having sequence identity or similarity to a DTC Protein, and truncations thereof as described herein (i.e. DTC Related Proteins) or dtc gene.

Truncated proteins may comprise peptides of between 3 and 70 amino acid residues, ranging in size from a tripeptide to a 70 mer polypeptide. The truncated proteins may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated proteins may have a carboxyl group, an amido group, a T-butyloxycarbonyl group, or a macromolecule including but not limited to lipid-fatty acid conjugates, polyethylene glycol, or carbohydrates at the carboxy terminal end.

The proteins of the invention may also include analogs of a DTC Protein, and/or truncations thereof as described herein, which may include, but are not limited to a DTC protein, containing one or more amino acid substitutions, insertions, and/or deletions. Amino acid substitutions may be of a conserved or non-conserved nature. Conserved amino acid substitutions involve replacing one or more amino acids of a DTC Protein amino acid sequence with amino acids of similar charge, size, and/or hydrophobicity characteristics. When only conserved substitutions are made the resulting analog is preferably functionally equivalent to a DTC Protein. Non-conserved substitutions involve replacing one or more amino acids of the DTC Protein amino acid sequence with one or more amino acids that possess dissimilar charge, size, and/or hydrophobicity characteristics.

One or more amino acid insertions may be introduced into a DTC Protein. Amino acid insertions may consist of single amino acid residues or sequential amino acids ranging from 2 to 15 amino acids in length.

Deletions may consist of the removal of one or more amino acids, or discrete portions from a DTC Protein sequence. The deleted amino acids may or may not be contiguous. The lower limit length of the resulting analog with a deletion mutation is about 10 amino acids, preferably 20 to 40 amino acids.

The proteins of the invention include proteins with sequence identity or similarity to a DTC Protein and/or truncations thereof as described herein. Such DTC Proteins include proteins whose amino acid sequences are comprised of the amino acid sequences of DTC Protein regions from other species that hybridize under selected hybridization conditions (see discussion of stringent hybridization conditions herein) with a probe used to obtain a DTC Protein. These proteins will generally have the same regions which are characteristic of a DTC Protein. Preferably a protein will have substantial sequence identity for example, about 65%, 70%, 75%, 80%, or 85% identity, preferably 90% identity, more preferably at least 95%, 96%, 97%, 98%, or 99% identity, and most preferably 98% identity with an amino acid sequence shown in in SEQ.ID.NO. 2 or 4. A percent amino acid sequence homology, similarity or identity is calculated as the percentage of aligned amino acids that match the reference sequence using known methods as described herein.

The invention also contemplates isoforms of the proteins of the invention. An isoform contains the same number and kinds of amino acids as a protein of the invention, but the isoform has a different molecular structure. Isoforms contemplated by the present invention preferably have the same properties as a protein of the invention as described herein.

The present invention also includes DTC Related Proteins conjugated with a selected protein, or a marker protein (see below) to produce fusion proteins. Additionally, immunogenic portions of a DTC Protein and a DTC Protein Related Protein are within the scope of the invention.

A DTC Related Protein of the invention may be prepared using recombinant DNA methods. Accordingly, the nucleic acid molecules of the present invention having a sequence which encodes a DTC Related Protein of the invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a recombinant expression vector of the invention containing a nucleic acid molecule of the invention, and the necessary regulatory sequences for the transcription and translation of the inserted protein-sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art. The necessary regulatory sequences may be supplied by the native DTC Protein and/or its flanking regions.

The invention further provides a recombinant expression vector comprising a DNA nucleic acid molecule of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a protein of the invention or a fragment thereof.

Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The recombinant expression vectors may also contain genes that encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g. a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BEK, HeLA, COS, MDCK, 293, 3T3, and W138 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a DTC Related Protein.

The proteins of the invention may also be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866)]. Procedures known in the art may be used to introduce a nucleic acid molecule of the invention encoding a DTC Related Protein into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the dtc gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant DTC Related Protein in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against a DTC Protein.

Proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a DTC Related Protein of the invention conjugated with other molecules, such as proteins, may be prepared by fusing, through recombinant techniques, the N-terminal or C-terminal of a DTC Related Protein, and the sequence of a selected protein or marker protein with a desired biological function. The resultant fusion proteins contain a DTC Protein fused to the selected protein or marker protein as described herein. Examples of proteins which may be used to prepare fusion proteins include immunoglobulins, glutathione-S-transferase (GST), hemagglutinin (HA), and truncated myc.

3. Antibodies

The tumor associated proteins, in particular DTC Related Proteins, can be used to prepare antibodies specific for the proteins. Antibodies can be prepared which bind a distinct epitope in an unconserved region of the protein. An unconserved region of the protein is one that does not have substantial sequence homology to other proteins. A region from a conserved region such as a well-characterized domain can also be used to prepare an antibody to a conserved region of a tumor associated protein. Antibodies having specificity for a tumor associated protein may also be raised from fusion proteins created by expressing fusion proteins in bacteria as described herein.

The invention can employ intact monoclonal or polyclonal antibodies, and immunologically active fragments (e.g. a Fab, (Fab)₂ fragment, or Fab expression library fragments and epitope-binding fragments thereof), an antibody heavy chain, and antibody light chain, a genetically engineered single chain Fv molecule (Ladner et al, U.S. Pat. No. 4,946,778), humanized antibodies, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

4. Applications

Nucleic acid molecules encoding tumor associated proteins (e.g. DTC Related Proteins), tumor associated proteins, and antibodies against the tumor associated proteins may be used in the prognostic and diagnostic evaluation of disorders involving the tumor associated proteins (e.g. DTC Related Proteins), and the identification of subjects with a predisposition to such disorders (Section 4.1.1 and 4.1.2). Methods for detecting nucleic acid molecules encoding tumor associated proteins and tumor associated proteins can be used to monitor disorders involving the proteins by detecting the proteins and nucleic acid molecules encoding the proteins. The applications of the present invention also include methods for the identification of compounds that modulate the biological activity of tumor associated proteins (e.g. DTC Related Proteins) (Section 4.2). The compounds, antibodies etc. may be used for the treatment of disorders involving a tumor associated protein (Section 4.3). It would also be apparent to one skilled in the art that the methods described herein may be used to study the developmental expression of tumor associated proteins (e.g DTC Related Proteins) and, accordingly, will provide further insight into the role of the proteins.

4.1 Diagnostic Methods

A variety of methods can be employed for the diagnostic and prognostic evaluation of disorders involving a tumor associated protein (e.g. DTC Related Protein), and the identification of subjects with a predisposition to such disorders. Such disorders include cancer, particularly ovarian and testicular cancer. Such methods may, for example, utilize nucleic acid molecules encoding tumor associated proteins (e.g. DTC Related Proteins), and fragments thereof, and antibodies directed against tumor associated proteins (e.g. DTC Related Proteins), including peptide fragments. In particular, the nucleic acids and antibodies may be used, for example, for: (1) the detection of the presence of mutations in genes encoding tumor associated proteins (e.g. dtc), or the detection of either over- or under-expression of tumor associated protein mRNA relative to a non-disorder state or the qualitative or quantitative detection of alternatively spliced forms of tumor associated protein transcripts which may correlate with certain conditions or susceptibility toward such conditions; and (2) the detection of either an over- or an under-abundance of tumor associated proteins relative to a non-disorder state or the presence of a modified (e.g., less than full length) tumor associated protein which correlates with a disorder state, or a progression toward a disorder state.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein may be performed by utilizing pre-packaged diagnostic kits comprising at least one specific tumor associated protein (e,g, dtc) nucleic acid or antibody described herein, which may be conveniently used, e.g., in clinical settings, to screen and diagnose patients and to screen and identify those individuals exhibiting a predisposition to developing a disorder.

Nucleic acid-based detection techniques are described, below, in Section 4.1.1. Peptide detection techniques are described, below, in Section 4.1.2. The samples that may be analyzed using the methods of the invention include those which are known or suspected to express nucleic acid molecules encoding tumor associated proteins or contain tumor associated proteins (e.g. DTC Related Proteins). The samples may be derived from a patient or a cell culture, and include but are not limited to biological fluids, tissue extracts, freshly harvested cells, and lysates of cells which have been incubated in cell cultures.

Oligonucleotides or longer fragments derived from any of the nucleic acid molecules encoding tumor associated proteins (e.g. DTC Related Proteins) may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

4.1.1 Methods for Detecting Nucleic Acid Molecules of the Invention

Those skilled in the art can construct nucleotide probes for use in the detection of nucleic acid sequences encoding tumor associated proteins in samples. Suitable probes include nucleic acid molecules based on nucleic acid sequences encoding at least 5 sequential amino acids from regions of a tumor associated protein (e.g. DTC Protein), preferably they comprise 15 to 30 nucleotides. A nucleotide probe may be labeled with a detectable substance such as a radioactive label which provides for an adequate signal and has sufficient half-life such as ³²p, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect genes, preferably in human cells, that encode tumor associated proteins (e.g. DTC Related Proteins). The nucleotide probes may also be useful in the diagnosis of disorders involving a tumor associated protein (e.g. DTC Related Protein); in monitoring the progression of such disorders; or monitoring a therapeutic treatment. In an embodiment, the probes are used in the diagnosis of, and in monitoring the progression of cancer, preferably ovarian and testicular cancer.

The probe may be used in hybridization techniques to detect genes that encode tumor associated proteins (e.g. DTC Related Proteins). The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving tumor associated protein gene (e.g. dtc) structure, including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations of a tumor associated protein gene (e.g. dtc). The polymorphisms may be used to identify individuals in families that are likely to carry mutations. If a polymorphism exhibits linkage disequalibrium with mutations in a tumor associated protein gene (e.g. dtc gene), it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of expression of tumor associated protein genes. For example, RNA may be isolated from a cell type or tissue known to express a tumor associated protein gene and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a disorder involving a tumor associated protein (e.g. DTC Related Protein) or gene.

The primers and probes may be used in the above described methods in situ i.e directly on tissue sections (fixed and/or frozen) of patient tissue obtained from biopsies or resections.

4.1.2 Methods for Detecting Tumor Associated Protein

Antibodies specifically reactive with a tumor associated protein (e.g. DTC Related Protein), or derivatives, such as enzyme conjugates or labeled derivatives, may be used to detect the proteins in various samples (e.g. biological materials). They may be used as diagnostic or prognostic reagents and they may be used to detect abnormalities in the level of protein expression, or abnormalities in the structure, and/or temporal, tissue, cellular, or subcellular location of a tumor associated protein (e.g. DTC Related Protein). Antibodies may also be used to screen potentially therapeutic compounds in vitro to determine their effects on disorders involving a tumor associated protein (e.g. DTC Related Protein), and other conditions. In vitro immunoassays may also be used to assess or monitor the efficacy of particular therapies. The antibodies of the invention may also be used in vitro to determine the level of expression of tumor associated protein genes in cells genetically engineered to produce a tumor associated protein (e.g. DTC Related Protein).

The antibodies may be used in any known immunoassays which rely on the binding interaction between an antigenic determinant of a tumor associated protein (e.g. DTC Related Protein) and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify a tumor associated protein (e.g DTC Related Protein) in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a tumor associated protein (e.g. DTC Related Protein), to localize it to particular cells and tissues, and to specific subcellular locations, and to quantitate the level of expression.

Cytochemical techniques known in the art for localizing antigens using light and electron microscopy may be used to detect a tumor associated protein (e.g DTC Related Protein). Generally, an antibody of the invention may be labeled with a detectable substance and a tumor associated protein (e.g. DTC Related Protein) may be localised in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a tumor associated protein (e.g. DTC Related Protein). By way of example, if the antibody having specificity against a tumor associated protein (e.g. DTC Related Protein) is a rabbit IgG antibody, the second antibody may be goat anti-rabbit gamma-globulin labeled with a detectable substance as described herein.

Where a radioactive label is used as a detectable substance, a tumor associated protein (e.g. DTC Related Protein) may be localized by radioautography. The results of radioautography may be quantitated by determining the density of particles in the radioautographs by various optical methods, or by counting the grains.

4.2 Methods for Identifying or Evaluating Substances/Compounds

The methods described herein are designed to identify substances that modulate the biological activity of a tumor associated protein (e.g. DTC Related Protein) including substances that bind to a tumor associated protein, or bind to other proteins that interact with a tumor associated protein, to compounds that interfere with, or enhance the interaction of a tumor associated protein and substances that bind to a tumor associated protein or other proteins that interact with a tumor associated protein. Methods are also utilized that identify compounds that bind to tumor associated protein gene (e.g. dtc) regulatory sequences.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], carbohydrates, oligosaccharides, polysaccharides, and small organic or inorganic molecules. The substance or compound may be an endogenous physiological compound or it may be a natural or synthetic compound.

Substances which modulate a tumor associated protein (e.g. DTC Related Protein) can be identified based on their ability to bind to a tumor associated protein. Therefore, the invention also provides methods for identifying substances which bind to a tumor associated protein (e.g. DTC Related Protein). Substances identified using the methods of the invention may be isolated, cloned and sequenced using conventional techniques. A substance that associates with a tumor associated protein may be an agonist or antagonist of the biological or immunological activity of a tumor associated protein.

The term “agonist” refers to a molecule that increases the amount of, or prolongs the duration of, the activity of the protein. The term “antagonist” refers to a molecule which decreases the biological or immunological activity of the protein. Agonists and antagonists may include proteins, nucleic acids, carbohydrates, or any other molecules that associate with a tumor associated protein.

Substances which can bind with a tumor associated protein (e.g. DTC Related Protein) may be identified by reacting a tumor associated protein with a test substance which potentially binds to a tumor associated protein, under conditions which permit the formation of substance-protein complexes and removing and/or detecting the complexes. The complexes can be detected by assaying for substance-protein complexes, for free substance, or for non-complexed protein. Conditions which permit the formation of substance-protein complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against a tumor associated protein (e.g. DTC Related Protein) or the substance, or labeled tumor associated protein, or a labeled substance may be utilized. The antibodies, proteins, or substances may be labeled with a detectable substance as described above.

A tumor associated protein (e.g. DTC Related Protein), or the substance used in the method of the invention may be insolubilized. For example, a tumor associated protein, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a tumor associated protein (e.g. DTC Related Protein), by assaying for an agonist or antagonist (i.e. enhancer or inhibitor) of the binding of a tumor associated protein with a substance which binds with a tumor associated protein. The basic method for evaluating if a compound is an agonist or antagonist of the binding of a tumor associated protein (e.g. DTC Related Protein) and a substance that binds to the protein, is to prepare a reaction mixture containing the protein and the substance under conditions which permit the formation of substance-protein complexes, in the presence of a test compound. The test compound may be initially added to the mixture, or may be added subsequent to the addition of the tumor associated protein and substance. Control reaction mixtures without the test compound or with a placebo are also prepared. The formation of complexes is detected and the formation of complexes in the control reaction but not in the reaction mixture indicates that the test compound interferes with the interaction of the tumor associated protein and substance. The reactions may be carried out in the liquid phase or the tumor associated protein, substance, or test compound may be immobilized as described herein. The ability of a compound to modulate the biological activity of a tumor associated protein may be tested by determining the biological effects on cells.

It will be understood that the agonists and antagonists i.e. inhibitors and enhancers that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the interaction of a tumor associated protein (e.g. DTC Related Protein) with a substance which is capable of binding to the tumor associated protein. Thus, the invention may be used to assay for a compound that competes for the same binding site of a tumor associated protein

The invention also contemplates methods for identifying compounds that bind to proteins that interact with a tumor associated protein (e.g. DTC Related Protein). Protein-protein interactions may be identified using conventional methods such as co-immunoprecipitation, crosslinking and co-purification through gradients or chromatographic columns. Methods may also be employed that result in the simultaneous identification of genes which encode proteins interacting with a tumor associated protein (e.g. DTC Related Protein). These methods include probing expression libraries with labeled tumor associated proteins.

Two-hybrid systems may also be used to detect protein interactions in vivo. Generally, plasmids are constructed that encode two hybrid proteins. A first hybrid protein consists of the DNA-binding domain of a transcription activator protein fused to a tumor associated protein (e.g. DTC Related Protein), and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the above-described methods. In particular, tumor associated proteins (e.g. DTC Related Proteins) fused to a glutathione-S-transferase may be used in the methods.

The reagents suitable for applying the methods of the invention to evaluate compounds that modulate a tumor associated protein (e.g. DTC Related Protein) may be packaged into convenient kits providing the necessary materials packaged into suitable containers. The kits may also include suitable supports useful in performing the methods of the invention.

4.3 Compositions and Treatments

The tumor associated proteins (e.g. DTC Related Proteins), substances or compounds that modulate tumor associated proteins, antibodies, and nucleic acid molecules encoding tumor associated proteins may be used for modulating the biological activity of a tumor associated protein (e.g. DTC Related Protein), and they may be used in the treatment of conditions such as cancer (particularly testicular and ovarian cancer). In particular, the proteins, antibodies, substances, compounds, and nucleic acid molecules, may be used to treat patients who have a tumor associated protein in, or on, their cancer cells. In an embodiment, the tumor associated protein is a DTC Related Protein.

A B form DTC Related Protein and substances or compounds identified by the methods of the invention that associate with or modulate a B form protein, antibodies specific for a B form protein and nucleic acid molecules encoding such B form proteins may also be used to modulate the biological activity of a B form protein involved in a disorder of the central nervous system. Such disorders include but are not limited to for example Alzheimer's disease, Parkinson's disease, Huntington's disease, demyelinating diseases, progressive spinal amyotrophy, trauma and ischemia resulting from stroke, and tumors of nerve tissue, epilepsy, glaucoma, and neurofibromatosis.

Accordingly, the proteins, substances, antibodies, peptides, and compounds may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to patients including humans, and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). For example, the compositions may be used in combination with anti-proliferative agents, antimicrobial agents, immunostimulatory agents, or anti-inflammatories. In particular, the compounds may be used in combination with anti-viral and/or anti-proliferative agents. The compositions of the invention may be administered concurrently, separately, or sequentially with other therapeutic agents or therapies.

Vectors derived from retroviruses, adenovirus, herpes or vaccinia viruses, or from various bacterial plasmids, may be used to deliver nucleic acid molecules to a targeted organ, tissue, or cell population. Methods well known to those skilled in the art may be used to construct recombinant vectors which will express antisense nucleic acid molecules. (See, for example, the techniques described in Sambrook et al (supra) and Ausubel et al (supra)).

The nucleic acid molecules comprising full length cDNA sequences and/or their regulatory elements enable a skilled artisan to use sequences encoding a tumor associated protein as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Genes encoding a tumor associated protein can be turned off by transfecting a cell or tissue with vectors which express high levels of a desired tumor associated protein-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a tumor associated protein, i.e., the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a tumor associated protein.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

An antibody against a tumor associated protein may be conjugated to chemotherapeutic drugs, toxins, immunological response modifiers, hematogenous agents, enzymes, and radioisotopes and used in the prevention and treatment of cancer (e.g. ovarian or testicular cancer). For example, an antibody against a tumor associated protein may be conjugated to toxic moieties including but not limited to ricin A, diphtheria toxin, abrin, modeccin, or bacterial toxins from Pseudomonas or Shigella. Toxins and their derivatives have been reported to form conjugates with antibodies specific to particular target tissues, such as cancer or tumor cells in order to obtain specifically targeted cellular toxicity (Moolten F. L. et al, Immun. Rev. 62:47-72, 1982, and Bernhard, M. I. Cancer Res. 43:4420, 1983).

Conjugates can be prepared by standard means known in the art. A number of bifunctional linking agents (e.g. heterobifunctional linkers such as N-succinimidyl-3-(2-pyridyldithio)propionate) are available commercially from Pierce Chemically Company, Rockford, Ill.

Administration of the antibodies or immunotoxins for therapeutic use may be by an intravenous route, although with proper formulation additional routes of administration such as intraperitoneal, oral, or transdermal administration may also be used.

A tumor associated protein may be conjugated to chemotherapeutic drugs, toxins, immunological response modifiers, enzymes, and radioisotopes using methods known in the art.

The invention also provides immunotherapeutic approaches for preventing or reducing the severity of a cancer. The clinical signs or symptoms of the cancer in a subject are indicative of a beneficial effect to the patient due to the stimulation of the subject's immune response against the cancer. Stimulating an immune response refers to inducing an immune response or enhancing the activity of immunoeffector cells in response to administration of a vaccine preparation of the invention. The prevention of a cancer can be indicated by an increased time before the appearance of cancer in a patient that is predisposed to developing cancer due for example to a genetic disposition or exposure to a carcinogenic agent. The reduction in the severity of a cancer can be indicated by a decrease in size or growth rate of a tumor.

Vaccines can be derived from a tumor associated protein (e.g. DTC Related Protein), peptides derived therefrom, or chemically produced synthetic peptides, or any combination of these molecules, or fusion proteins or peptides thereof. The proteins, peptides, etc. can be synthesized or prepared recombinantly or otherwise biologically, to comprise one or more amino acid sequences corresponding to one or more epitopes of a tumor associated protein. Epitopes of a tumor associated protein will be understood to include the possibility that in some instances amino acid sequence variations of a naturally occurring protein or polypeptide may be antigenic and confer protective immunity against cancer or anti-tumorigenic effects. Sequence variations may include without limitation, amino acid substitutions, extensions, deletions, truncations, interpolations, and combinations thereof. Such variations fall within the scope of the invention provided the protein containing them is immunogenic and antibodies against such polypeptide cross-react with naturally occurring tumor associated proteins to a sufficient extent to provide protective immunity and/or anti-tumorigenic activity when administered as a vaccine.

The proteins, peptides etc, can be incorporated into vaccines capable of inducing an immune response using methods known in the art. Techniques for enhancing the antigenicity of the proteins, peptides, etc. are known in the art and include incorporation into a multimeric structure, binding to a highly immunogenic protein carrier, for example, keyhole limpet hemocyanin (KLH), or diptheria toxoid, and administration in combination with adjuvants or any other enhancer of immune response.

Vaccines may be combined with physiologically acceptable media, including immunologically acceptable diluents and carriers as well as commonly employed adjuvants such as Freund's Complete Adjuvant, saponin, alum, and the like.

It will be further appreciated that anti-idiotype antibodies to antibodies to tumor associated proteins described herein are also useful as vaccines and can be similarly formulated.

The administration of a vaccine in accordance with the invention, is generally applicable to the prevention or treatment of cancers including ovarian and testicular cancer.

The administration to a patient of a vaccine in accordance with the invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. The cancer immunotherapy in accordance with the invention would be a preferred treatment for the prevention and/or treatment of cancer, since the side effects involved are substantially minimal compared with the other available treatments e.g. surgery, chemotherapy, radiation therapy. The vaccines have the potential or capability to prevent cancer in subjects without cancer but who are at risk of developing cancer.

The activity of the proteins, substances, compounds, antibodies, nucleic acid molecules, agents, and compositions of the invention may be confirmed in animal experimental model systems. Therapeutic efficacy and toxicity may be determined by standard pharmaceutical procedures in cell cultures or with experimental animals, such as by calculating the ED₅₀ ( the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

4.4 Other Applications

The nucleic acid molecules encoding DTC Related Proteins may be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a DTC Related Protein. Cells, tissues, and non-human animals lacking in expression or partially lacking in expression of a nucleic acid molecule or gene encoding a DTC Related Protein may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded polypeptide using the methods described herein. Such cells may then be fused to embryonic stem cells to generate transgenic non-human animals deficient in a polypeptide of the invention. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras.

Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a gene encoding a DTC Related Protein. In an embodiment the invention provides a transgenic non-human mammal all of whose germ cells and somatic cells contain a recombinant expression vector that inactivates or alters a gene encoding a DTC Related Protein resulting in a DTC Related Protein associated pathology. Further the invention provides a transgenic non-human mammal which doe not express or has altered (e.g. reduced) expression of a DTC Related Protein of the invention. In an embodiment, the invention provides a transgenic non-human mammal which doe not express or has reduced expression of a DTC Related Protein of the invention resulting in a DTC Related Protein associated pathology. A DTC Related Protein associated pathology refers to a phenotype observed for a DTC Related Protein homozygous or heterozygous mutant.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with a DTC Related Protein, preferably a DTC Related Protein associated pathology, comprising:

(a) administering the agent to a transgenic non-human animal of the invention; and

(b) determining whether said agent reduces or inhibits the pathology (e.g. DTC Related Protein associated pathology) in the transgenic non-human animal relative to a transgenic non-human animal of step (a) which has not been administered the agent.

The agent may be useful in the treatment and prophylaxis of conditions such as cancer as discussed herein. The agents may also be incorporated in a pharmaceutical composition as described herein.

The following non-limiting example is illustrative of the present invention:

### EXAMPLE

### Materials and Methods

Immunoscreening of an Ovarian Carcinoma cDNA Expression Library with Ascites from Ovarian Cancer Patients.

The Uni-ZAP™ XR premade ovarian carcinoma cDNA expression library was purchased from Stratagene (La Jolla, Calif.). Ovarian cancer ascites fluids were pooled from 5 different primary ovarian cancer patients. The cDNA library was plated on NZY agar plates at a density of 500 clones/15 cm plate. The plates were incubated at 42° C. for 4 hours to allow plaques to develop. Nitrocellulose filters soaked with IPTG were then laid on top of the plaques and incubated at 37° C. for 4 hours to transfer the plaques onto the membranes. The filters were blocked with 5% non-fat dried milk/PBS-T overnight, at 4° C. The PBS-T buffer contained 80 mM sodium orthophosphate, 20 mM sodium dihydrogen orthophosphate, 100 mM sodium chloride, and 0.1% Tween-20. To screen the library, the ascites were diluted 1:100 in 5% non-fat dried milk/PBS-T. The diluted ascites were first incubated with E. coli phage lysate (from Stratagene) for 2 hours at room temperature to minimize the cross-reaction between the autoantibodies and the bacterial/phage proteins. Nitrocellulose filters were incubated with this preabsorbed ascites for 2 hours at room temperature to identify the cellular proteins that react with the autoantibodies in ascites. Following probing with the ascites, the filters were washed and further treated with goat anti-human IgG conjugated with alkaline phosphatase (Jackson Immunoresearch Laboratories, West Grove, Pa.) at 1:2000 dilution, in the blocking buffer for 1 hour at room temperature, then proceeded to chemiluminescence detection with dioxetane-based substrate (Diagnostic Products Corporation, Los Angeles, Calif.). The plaques exhibiting immunoreactivity were excised from the plates and the phages were converted into the pBluescript phagemid form by in vivo excision with Exassist™ helper phage following the manufacturer's instructions (Stratagene). The excised phagemids were purified and subjected to automated DNA sequencing with M13 forward and reverse primers. The insert sequences were compared to the known sequences in the Genbank database (http//www.ncbi.nlm.nih.gov) with the BLASTN alignment algorithm (14).

Northern Blot Analysis

The human multiple tissue blot was obtained from OriGene Technologies, Inc. (Rockville, Md.). Anti-sense RNA probe labelled with digoxigenin was prepared by in vitro transcription using the T7 RNA polymerase kit (Roche Molecular Systems, Laval, Quebec, Canada) following the manufacturer's instructions. The blot was hybridized with the RNA probe in Ultrahyb™ (OriGene) hybridization buffer overnight at 68° C., then subsequently washed with 2×SSC, 0.1% SDS and 0.2×SSC, 0.1% SDS at 68° C. for 15 minutes. To detect the signal, the blot was incubated with alkaline phosphatase-conjugated anti-digoxigenin antibody (Roche) at 1:2000 dilution for one hour, then proceeded to chemiluminescence detection with dioxetane-based substrate.

Human Tissue Total RNA and Cancerous/Normal Testicular tissue Total RNA.

Human Tissue total RNA was purchased from Clontech Laboratories, Inc. (Palo Alto, Calif.). 14 pairs of cancerous and normal testicular tissues were obtained from 14 patients who had undergone radical orchiectomy for testicular tumors at the Charite Hospital, Germany. All patients had a histologically-confirmed diagnosis of primary testicular cancer and received no treatment before surgery. Matched cancerous and normal tissues were obtained from the cancerous and non-cancerous parts of the same testis. Of these 14 tumors, 6 were seminomas and 8 non-seminomas. Tissue specimens were homogenized and total RNA was extracted using RNeasy Mini Spin columns (QIAGEN Inc., Valencia, Calif.) following the manufacturer's instructions. Total RNA concentration and quality were determined spectrophotometrically.

Reverse Transcriptase Polymerase Chain Reaction (RT-PCR)

2 μg of total RNA was converted to cDNA with Superscript™ Preamplification Kit (Gibco BRL, Gaithersburg, Md.), according to the manufacturer's recommendations. The final volume was 20 μL. The PCR primers are as follows, forward, 5′-CTTGCTTGTATCATGGGATAC-3′ (SEQ ID NO: 24); reverse, 5′-CCAATTCAACATGCAGATATC-3′(SEQ ID NO: 25). PCR was carried out in a 20 μl reaction mixture, containing 1 μl cDNA, 10 mM Tris-HCl (pH 8.3), 50 mM KCl, 1.5 mM MgCl₂, 200 μM dNTPs (deoxynucleoside triphosphates), 100 ng primers and 2.5 units of HotstarTaq™ DNA polymerase (QIAGEN Inc.) on a Perkin-Elmer 9600 thermal cycler. The PCR conditions were 94° C. for 15 minutes to activate the polymerase, following by 30 cycles of 94° C. for 30 seconds, 58° C. for 1 minute, 72° C. for 1 minute and a final extension 72° C. for 10 minutes. The PCR products were then separated on a 2.5% agarose gel, stained with ethidium bromide, and visualised under UV light.

Chromosomal Localization of the Novel Gene by Fluorescence in-situ Hybridization (FISH).

A BAC clone (clone identification: 19F13) containing the novel gene was labeled with biotin by nick translation. This BAC clone was identified by screening a genomic BAC library (15) with the initial excised phagemid, as described elsewhere (16). The regional assignment of the BAC clone was determined by FISH to normal human lymphocyte chromosomes counterstained with propidium iodide and 4′, 6-diamidin-2-phenylindol-dihydrochloride (DAPI). Biotinylated probe was detected with avidin-fluorescein isothiocyanate (FITC), followed by biotinylated anti-avidin antibody and avidin-FITC. Images of metaphase preparations were captured by a thermoelectrically cooled charge coupled camera (Photometrics, Tucson, Ariz.). Separate images of DAPI banded chromosomes and of FITC targeted chromosomes were obtained, pseudo colored blue (DAPI) and yellow (FITC) and merged electronically using Adobe PhotoshopA 3.0 software. The band assignment was determined by measuring the fractional chromosome length and by analyzing the banding pattern generated by the DAPI counterstained image.

### Results

Identification of a Novel Ovarian Carcinoma Immunoreactive Antigen by Immunoscreening of an Ovarian Carcinoma cDNA Expression Library with Ovarian Cancer Ascites.

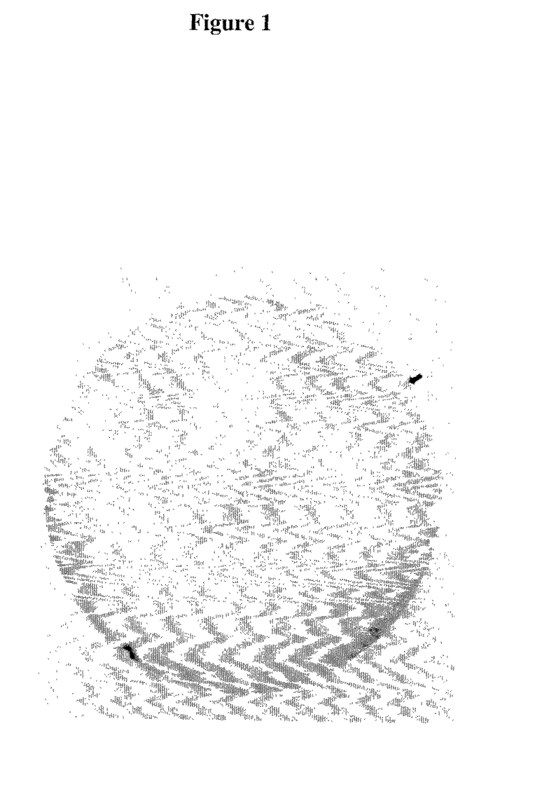

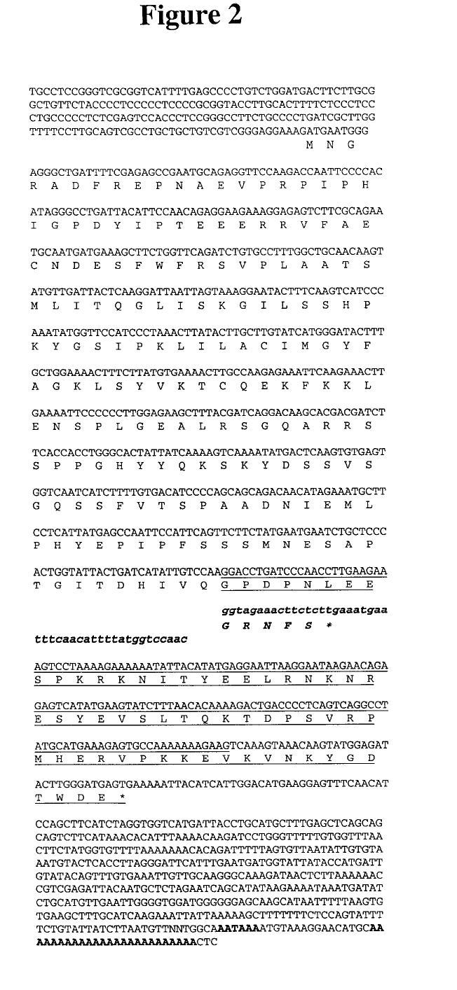

In order to identify cellular proteins that trigger immune responses in ovarian cancer, a λ ZAP phage ovarian carcinoma cDNA expression library was screened with ascites from ovarian cancer patients. An example of screened positive clones is shown in FIG. 1. Positive clones were excised from the plates and the λ ZAP phages were converted into the phagemid form. The phagemids were then sequenced with vector-specific primers. The identity of the insert sequences was examined using the BLASTN program and the Genbank databases. Thirteen proteins were identified to have immunoreactivities in ovarian cancer (Table 1). Twelve are known proteins. One immunoreactive clone was identified with insert sequence not matching with any known gene, suggesting that it encoded for a novel gene. The length of this insert sequence was about 800 bp. The polyA tail and the polyadenylation signal were found in the 3′ end of this sequence (FIG. 2).

Identification of the Full-length cDNA Sequence of the Novel Gene

In order to obtain the full-length cDNA sequence of the novel gene, the Genbank EST database was searched with the initial 800 bp sequence and a number of matching ESTs were found. With ESTs AA099273, W44679, and AA332589, a 1.4 kb contiguous sequence was constructed (FIG. 2). To obtain more sequence, a testis cDNA library (OriGene Technologies, Inc.) was screened twice, 6 clones were found to contain this new gene, but when these clones were sequenced, no more new sequence was identified.

Determining the Transcript Size of the Novel Gene in Human Tissues

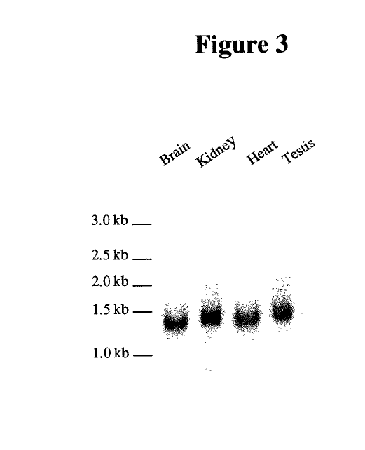

To assess the transcript size of the novel gene and determine whether the 1.4 kb cDNA sequence obtained is full length, northern blot analysis was performed. An anti-sense RNA probe was prepared using in vitro transcription with the initial excised phagemid as a template. This RNA probe was then hybridized to a blot containing 2 μg of polyA RNA. This method detected a distinct band of about 1.4 kb in length. No other band was identified (FIG. 3). This result suggested that the 1.4 kb cDNA sequence, very likely represented the full-length cDNA of the novel gene.

Tissue Specific Expression of the Novel Gene

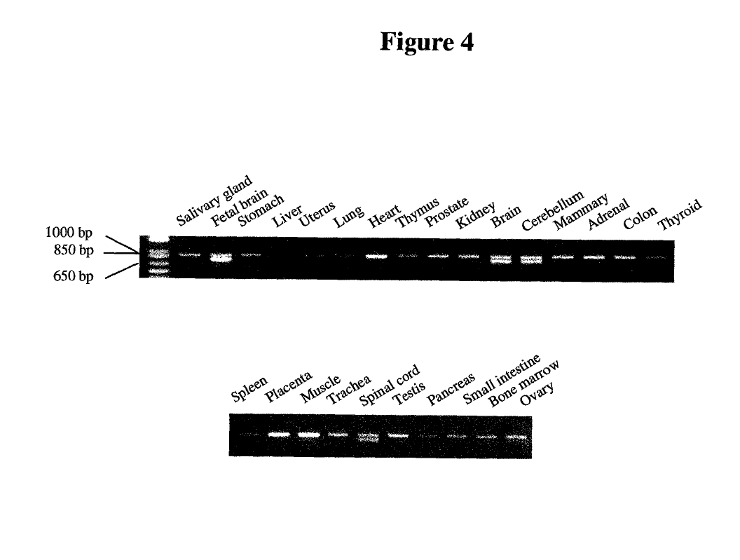

The tissue expression pattern of the novel gene was investigated by RT-PCR. As shown in FIG. 4, it is highly expressed in all tissues tested, except liver. Two PCR products were detected on the agarose gel, with approximate length of 850 bp and 700 bp. By cloning and sequencing of these two bands, the longer band was shown to have the same sequence as the cDNA sequence already obtained. The sequence of the smaller size band revealed that it represented an alternatively spliced transcript. The longer transcript is referred to as “alternatively spliced form A” and the shorter one as “alternatively spliced form B”. It is worth mentioning that only in the brain tissue, spinal cord, and cerebellum, the expression of alternatively spliced form B is more predominant than form A, whereas, in the other tissues, form A is the only form expressed (FIG. 4).

The Identified Transcripts Encode for Novel Proteins

One open reading frame was identified within the full-length cDNA sequence of the novel gene. For alternatively spliced form A, it encodes a protein of 245 amino acids with molecular mass of 27.6 kDa. Form B encodes for a truncated protein of 208 amino acids with molecular mass of 20.7 kDa (FIG. 2). In order to determine the identity of these proteins, the BLASTP, BLASTP+BEAUTY (http:/www.dot.imgen.bcm.tmc.edu.9331) and BLOCKS protein motif (http:fwww.blocks.fhctc.org) programs were used to search the databases. One putative protein submitted by the Drosophila genome project (Genbank accession number: AAF46923) was found to have 30% identity and 46% similarity at the amino acid sequence level with these novel proteins. The identity and function of this protein are still not known. No other significant homologies were found with any known proteins.

Genomic Organization of the Novel Gene

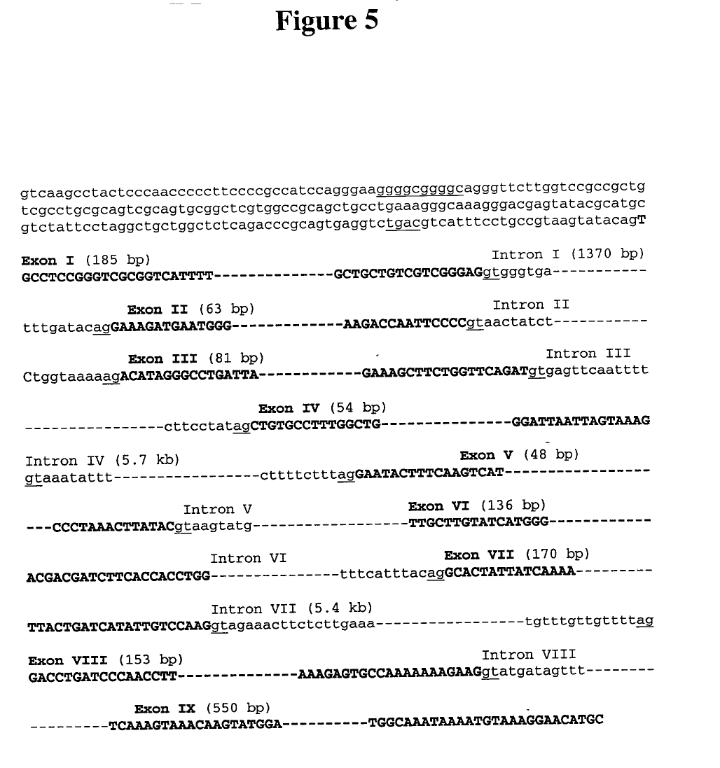

To establish the genomic organization of the new gene, the Genbank database was searched with the cDNA sequence. Two BAC clones, 19F13 (accession number: AC011956) and 15P24 (accession number: AC011977.1) were found to contain the majority of the gene sequence. By aligning the cDNA sequence with the BACs, the intron/exon organization of this gene was established. As shown in FIG. 5, this gene consists of eight introns and nine exons Exon VI and exon IX sequences were missing from the BACs and were considered continuous exons, but the splice sites could not be established). Exon I is untranslated. Among the eight introns, full-length sequences are given for introns I, IV, and VII. For the rest of the introns, the partial sequence is provided. The intron-exon splice junctions are completely conserved, following the rule that the “GT” is in the splice donor site and the “AG” is in the splice acceptor site (17). Within the 200 bp region upstream from the cDNA start site, although a typical “TATA box” was not identified, the sequence in this region is GC rich, indicating that it may harbor the CpG island. In addition, a consensus SP1 binding site “GGGGCGGGGC” (SEQ ID NO: 26) (18) and a CREB binding site “TGAC” (19) were found within this region. These findings suggest that this region represents the potential promoter of the novel gene.

Chromosomal Localization of the Novel Gene

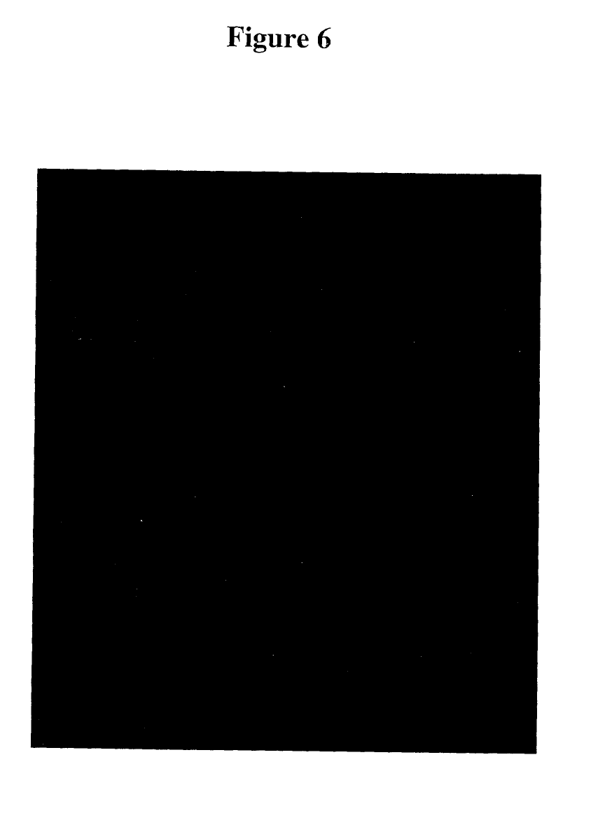

To determine the chromosomal localization of this new gene, fluorescence in situ hybridization was performed using the BAC clone 19F13 as a probe. It was mapped to chromosome 4p11 (FIG. 6).

The Expression of the Novel Gene is Down-Regulated in Testicular Tumors

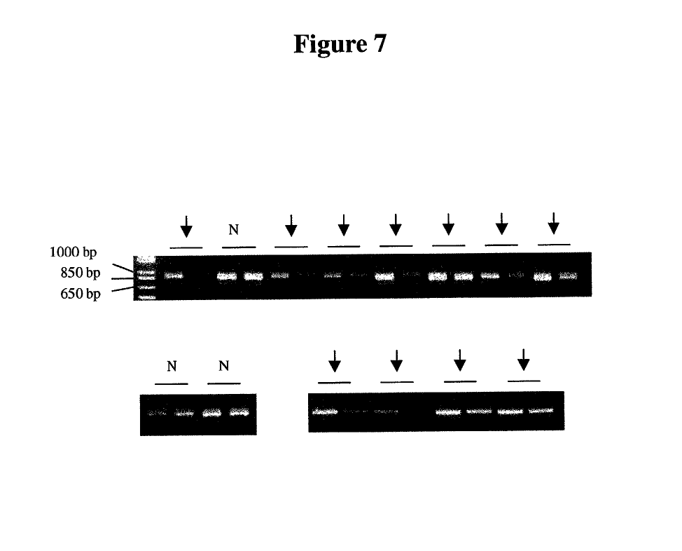

Although this novel gene was identified by screening an ovarian cancer library, its expression is widespread in many tissues. As already mentioned, six clones containing this gene were identified by screening a testis cDNA library. Therefore, the potential role of this novel gene in testicular cancer was investigating by examining whether its expression is different between testicular tumors and normal testicular tissues, by RT-PCR. 14 pairs of cancerous/normal testicular tissues were examined. The expression level of this gene was lower in the tumors compared to their adjacent normal tissues in 11 pairs. For the other 3 pairs, there was no change (FIG. 7). These results suggest that the expression of this gene is down-regulated in the majority of testicular tumors. This new gene is named DTC (down-regulated in testicular cancer).

### Discussion

The method of immunoscreening of an ovarian carcinoma cDNA expression library with ovarian cancer ascites fluid was used to identify cellular components that may trigger immune responses in ovarian cancer. One immunoreactive clone containing the partial sequence of a novel gene was identified. The full-length sequence was cloned and a new gene, tentatively named DTC (down-regulated in testicular cancer) was characterized. The encoded protein has no significant homology to any other known proteins and its expression is down-regulated in testicular tumors.

There are two alternatively spliced forms of the DTC gene. Interestingly, the tissue distribution of these two forms is quite different. The longer form A is expressed in almost all human tissues tested (FIG. 4). The shorter form B, is only expressed abundantly in the central nervous system, including brain, cerebellum, and spinal cord. The DTC gene is down-regulated in testicular tumors. Testicular cancer is the most common malignancy of young males, but its etiology is still not clear (25). The down-regulation of the DTC gene indicates that this gene may play a role in tumor formation or progression. Its down-regulation may due to direct suppression (as a result of mutations in the DTC gene) or alteration of regulatory elements. These mechanisms have been proposed to explain the down-regulation of many tumor suppressor genes in cancer (26).

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

TABLE 1
Proteins identified by immunoscreening of an ovarian carcinoma
cDNA expression library with ovarian cancer ascites.

| Clone | | |
|---|---|---|
| name | Identity (Genbank accession number) | Functional significance |
| 841 | Ribosomal protein S18 (X69150) | Protein translation |
| 816-3 | Heat shock protein (90kda) (M16660) | Molecular chaperone |
| 816-4 | JK-recombination signal binding protein | Transcription factor |
| | (L07872) | |
| 816-5 | Ribonucleoprotein H1 (L22009) | mRNA processing |
| 816-6 | RAN binding protein 7 (AF098799) | Mediate nuclear |
| | | import of ribosomal |
| | | proteins |
| 816-7 | TG-interacting factor (X89750) | Transcription factor |
| 816-8 * | Unknown, EST AA099273 | Unknown |
| 816-9 | Eukaryotic translation initiation factor | Protein translation |
| | 3, p40 (U54559) | |
| 816-10 | Human amyloid precursor protein- | Cleave beta-amyloid |
| | binding protein 1 (U50939) | protein precursor |
| 1210-1 | Ribosomal protein L8 (NM000973) | Protein translation |
| 1210-2 | CDC23 (cell division 23), (NM004661) | Cell devision |
| 1210-3 | IQ motif containing GTPase activating | Cell-cell adhesion |
| | protein 1(IQGAP1) (NM003870) | |
| 1210-4 | Ribosomal protein L3 (Nm000967) | Protein translation |

* This is the novel gene identified.

### FULL CITATIONS FOR REFERENCES REFERRED TO IN THE SPECIFICATION

1. Fraumeni, J. F., Hoover, R. N., Devesa, S. S., and Kinlen, L. J. Epidemiology of Cancer. In: V. T. Devita, S. Hellman, and S. A. Rosenberg (eds.), Cancer: principles and practice of oncology, 3 edition, Vol. 2, pp. 196-235. Philadelphia: Lippincott, 1994.

2. Zouali, M. Trigger mechanisms of autoantibody production, Clin Exp Rheumatol. 12 Suppl 11: S33-6, 1994.

3. Tan, E. M. Autoantibodies in pathology and cell biology, Cell. 67: 841-2, 1991.

4. Abrams, M. B., Bednarek, K. T., Bogoch, S., Bogoch, E. S., Dardik, H. J., Dowden, R., Fox, S. C., Goins, E. E., Goodfried, G., Herrman, R. A., and et al. Early detection and monitoring of cancer with the anti-malignin antibody test, Cancer Detect Prev. 18: 65-78, 1994.

5. Amagai, M., Klaus-Kovtun, V., and Stanley, J. R. Autoantibodies against a novel epithelial cadherin in pemphigus vulgaris, a disease of cell adhesion, Cell. 67: 869-77, 1991. 6. Angelopoulou, K. and Diamandis, E. P. Detection of the TP53 tumor suppressor gene product and p53 auto-antibodies in the ascites of women with ovarian cancer, Eur J Cancer. 33: 115-21, 1997.

7. Angelopoulou, K., Diamandis, E. P., Sutherland, D. J., Kellen, J. A., and Bunting, P. S. Prevalence of serum antibodies against the p53 tumor suppressor gene protein in various cancers, Int J Cancer. 58: 480-7, 1994.

8. Angelopoulou, K. and Diamandis, E. P. Autoantibodies against the p53 tumor suppressor gene product quantified in cancer patient serum with time-resolved immunofluorometry, Cancer J. 6: 315-321, 1993.

9. Hassapoglidou, S. and Diamandis, E. P. Antibodies to the p53 tumor suppressor gene product quantified in cancer patient serum with a time-resolved immunofluorometric technique, Clin Biochem. 25: 445-9, 1992.

10. Laurent-Puig, P., Lubin, R., Semhoun-Ducloux, S., Pelletier, G., Fourre, C., Ducreux, M., Briantais, M. J., Buffet, C., and Soussi, T. Antibodies against p53 protein in serum of patients with benign or malignant pancreatic and biliary diseases, Gut. 36: 455-8, 1995.

11. O'Brien, T. J., Tanimoto, H., Konishi, I., and Gee, M. More than 15 years of CA 125: what is known about the antigen, its structure and its function, Int J Biol Markers. 13: 188-95, 1998.

12. Rosenthal, A. N. and Jacobs, I. J. The role of CA 125 in screening for ovarian cancer, Int J Biol Markers. 13: 216-20, 1998.

13. Mazurek, A., Niklinski, J., Laudanski, T., and Pluygers, E. Clinical tumour markers in ovarian cancer, Eur J Cancer Prev. 7.-23-35, 1998.

14. Altschul, Stephen, F., Madden, T. L., Schaffer, A. A., Zhang, J., Zhang, Z., Miller, W., and Lipman , D. J. Gapped BLAST and PSI-BLAST: a new generation of protein database search programs, Nucleic Acids Res. 25: 3389-3402, 1997.

15. Shizuya, H., Birren, B., Kira, U. J., Mancino, V., Slepak, T., Tachiiri, Y., and Simon, M. Cloning and stable maintenance of 300-kilobase-pair fragments of human DNA in Escherichia coli using an F-factor-based vector., Proc. Natl. Acad. Sci. USA. 89: 8794-8797, 1992.

16. Luo, L., Herbrick, J. A., Scherer, S. W., Beatty, B., Squire, J., and Diamandis, E. P. Structural characterization and mapping of the normal epithelial cell-specific 1 gene, Biochem Biophys Res Commun. 247. 580-6,1998.

17. Mount, S. M. A catalogue of splice junction sequences, Nucleic Acids Res. 10: 459-72, 1982.

18. Briggs, M. R., Kadonaga, J. T., Bell, S. P., and Tjian, R. Purification and biochemical characterization of the promoter-specific transcription factor, Sp1, Science. 234: 47-52, 1986.

19. Benbrook, D. M. and Jones, N. C. Different binding specificities and transactivation of variant CRE's by CREB complexes, Nucleic Acids Res. 22: 1463-1469, 1994.

20. Jager, D., Stockert, E., Scanlan, M. J., Gure, A. O., Jager, E., Knuth, A., Old, L. J., and Chen, Y. T. Cancer-testis antigens and ING1 tumor suppressor gene product are breast cancer antigens: characterization of tissue-specific ING1 transcripts and a homologue gene, Cancer Res. 59: 6197-204, 1999.

21. Lin, B., White, J. T., Ferguson, C., Bumgarner, R., Friedman, C., Trask, B., Ellis, W., Lange, P., Hood, L., and Nelson, P. S. PART-1: a novel human prostate-specific, androgen-regulated gene that maps to chromosome 5q12, Cancer Res. 60: 858-63, 2000.

22. Sahin, U., Tureci, O., Schmitt, H., Cochlovius, B., Johannes, T., Schmits, R., Stenner, F., Luo, G., Schobert, I., and Pfreundschuh, M. Human neoplasms elicit multiple specific immune responses in the autologous host, Proc. Natl. Acad. Sci. U S A. 92: 11810-3, 1995.

23. Sahin, U., Tureci, O., and Pfreundschuh, M. Serological identification of human tumor antigens, Curr. Opin. Immunol. 9: 709-716, 1997.

24. Old, L. J. and Chen, Y. T. New paths in human cancer serology, J. Exp. Med. 187: 1163-1167, 1998.

25. Einhorn, L. Cancer of the Testes. In: V. T. Devita, S. Hellman, and S. A. Rosenberg (eds.), Cancer: principles and practice of oncology, 3 edition, Vol. 2, pp. 1071-1092. Philadelphia: Lippincott, 1994.

26. Lee, S. W., Tomasetto, C., and Sager, R. Positive selection of candidate tumor-suppressor genes by subtractive hybridization, Proc. Natl. Acad. Sci. U S A. 88: 2825-9, 1991.

We claim:

1. An isolated nucleic acid molecule of at least 30 nucleotides which hybridizes to one or more of SEQ. ID. NO. 1, 3, and 5 to 21, or the complement of one or more of SEQ ID NO. 1, 3, and 5 to 21 under stringent hybridization conditions.

2. An isolated nucleic acid molecule as claimed in claim 1 which comprises:
(i) a nucleic acid sequence encoding a protein having substantial sequence identity with an amino acid sequence of SEQ. ID. NO. 2 or 4;
(ii) a nucleic acid sequence encoding a protein comprising an amino acid sequence of SEQ. ID. NO. 2 or 4;
(iii) nucleic acid sequences complementary to (i) or (ii);
(iv) a degenerate form of a nucleic acid sequence of (i) or (ii);
(v) a nucleic acid sequence capable of hybridizing under stringent conditions to a nucleic acid sequence in (i), (ii) or (iii);
(vi) a nucleic acid sequence encoding a truncation, an analog, an allelic or species variation of a protein comprising an amino acid sequence of SEQ. ID. NO. 2 or 4; or
(vii) a fragment, or allelic or species variation of (i), (ii) or (iii).

3. An isolated nucleic acid molecule as claimed in claim 1 which comprises:
(i) a nucleic acid sequence comprising the sequence of one or more of SEQ.ID.NO. 1, 3, and 5 to 21 wherein T can also be U;
(ii) nucleic acid sequences complementary to (i), preferably complementary to the full nucleic acid sequence of one or more of SEQ.ID.NO. 1, 3, and 5 to 21;
(iii) a nucleic acid capable of hybridizing under stringent conditions to a nucleic acid of (i) or (ii) and preferably having at least 18, 20, 25, 30, 35, or 40 nucleotides; or
(iv) a nucleic acid molecule differing from any of the nucleic acids of (i) to
(iii) in codon sequences due to the degeneracy of the genetic code.

4. A vector comprising a nucleic acid molecule of claim 2.

5. A host cell comprising a nucleic acid molecule of claim 2.

6. A method for preparing a DTC protein comprising:
(a) transferring a vector as claimed in claim 4 into a host cell;
(b) selecting transformed host cells from untransformed host cells;
(c) culturing a selected transformed host cell under conditions which allow expression of the protein; and
(d) isolating the protein.

7. A DTC Protein comprising an amino acid sequence of SEQ ID NO: 2 or 4.

8. An A form of a DTC Protein as claimed in claim 7 comprising the amino acid sequence of SEQ ID NO: 2.

9. A B form of a DTC Protein as claimed in claim 7 comprising the amino acid sequence of SEQ ID NO: 4.

10. An antibody having specificity against an epitope of a protein as claimed in claim 7.

11. An antibody as claimed in claim 10 labeled with a detectable substance and used to detect a DTC Protein in biological samples, tissues, and cells.

12. A method for detecting a nucleic acid molecule as claimed in claim 2 in a biological sample comprising the steps of:
(a) hybridizing a nucleic acid molecule of claim 2 to nucleic acids of the biological sample, thereby forming a hybridization complex; and
(b) detecting the hybridization complex wherein the presence of the hybridization complex correlates with the presence of a nucleic acid molecule encoding a DTC Protein in the biological sample.

13. A method for diagnosing and monitoring cancer mediated by a tumor associated protein by determining the presence of a nucleic acid molecule encoding the tumor associated protein or the presence of the tumor associated protein wherein the tumor associated protein is selected from the group consisting of ribosomal protein S18 (Genbank X69150), heat shock protein (90 kda) (Genbank M16660), JK-recombination signal binding protein (Genbank (L07872), ribonucleoprotein H1 (Genbank L22009), RAN binding protein 7 (AF098799), TG-interacting factor (Genbank X89750), eukaryotic translation initiation factor 3, p40 (Genbank U54559), human amyloid percursor protein-binding protein 1 (Genbank U50939), ribosomal protein L8 (NM000973), CDC23 (Cell division 23) (Genbank NM004661), IQ motif containing GTPase activating protein 1 (IQGAP1) (Genbank NM003870), ribosomal protein L3 (Genbank NM000967), and a protein as claimed in claim 7.

14. A method as claimed in claim 13 wherein the condition is breast cancer.

15. A method for preventing or treating a condition mediated by a tumor associated protein selected from the group consisting of ribosomal protein S18 (Genbank X69150), heat shock protein (90 kda) (Genbank M16660), JK-recombination signal binding protein (Genbank (L07872), ribonucleoprotein H1 (Genbank L22009), RAN binding protein 7 (AF098799), TG-interacting factor (Genbank X89750), eukaryotic translation initiation factor 3, p40 (Genbank U54559), human amyloid percursor protein-binding protein 1 (Genbank U50939), ribosomal protein L8 (NM000973), CDC23 (Cell division 23) (Genbank NM004661), IQ motif containing GTPase activating protein 1 (IQGAP1) (Genbank NM003870), ribosomal protein L3 (Genbank NM000967), and a protein as claimed in claim 7, comprising administering an effective amount of an antibody specific for the tumor associated protein.

16. A vaccine to prevent cancer and/or to treat cancer comprising a protein selected from the group consisting of ribosomal protein S18 (Genbank X69150), heat shock protein (90 kda) (Genbank M16660), JK-recombination signal binding protein (Genbank (L07872), ribonucleoprotein H1 (Genbank L22009), RAN binding protein 7 (AF098799), TG-interacting factor (Genbank X89750), eukaryotic translation initiation factor 3, p40 (Genbank U54559), human amyloid percursor protein-binding protein 1 (Genbank U50939), ribosomal protein L8 (NM000973), CDC23 (Cell division 23) (Genbank NM004661), IQ motif containing GTPase activating protein 1 (IQGAP1) (Genbank NM003870), ribosomal protein L3 (Genbank NM000967), and a protein as claimed in claim 7, peptides derived therefrom, or synthetic peptides thereof, or any combination of these molecules.

17. A method to prevent cancer or to treat cancer in subjects who have a tumor associated protein on their cells comprising administering a vaccine for stimulating or enhancing in the subjects antibodies directed against the tumor associated protein wherein the tumor associated protein is selected from the group consisting of ribosomal protein S18 (Genbank X69150), heat shock protein (90 kda) (Genbank M16660), JK-recombination signal binding protein (Genbank (U)7872), ribonucleoprotein H1 (Genbank L22009), RAN binding protein 7 (AF098799), TG-interacting factor (Genbank X89750), eukaryotic translation initiation factor 3, p40 (Genbank U54559), human amyloid percursor protein-binding protein 1 (Genbank U50939), ribosomal protein L8 (NM000973), CDC23 (Cell division 23) (Genbank NM004661), IQ motif containing GTPase activating protein 1 (IQGAP1) (Genbank NM003870), ribosomal protein L3 (Genbank NM000967), and a protein as claimed in claim 7.

18. A method for preventing patients from having tumors prior to their occurrence comprising administering a vaccine as claimed in claim 16.

19. A method for stimulating or enhancing in a subject production of antibodies directed against a tumor associated protein comprising administering to the subject a vaccine as claimed in claim 16 in a dose effective for stimulating or enhancing production of the antibodies.

20. A methods for treating, preventing, or delaying recurrence of cancer comprising administering to the subject a vaccine as claimed in claim 16 in a dose effective for treating, preventing, or delaying recurrence of cancer.
